(19) United States
(12) Patent Application Publication
(10) Pub. No.: US 2023/0149122 A1
(43) Pub. Date: May 18, 2023

(54) STIMULATION ARRANGEMENT AND METHOD OF OPERATING SUCH STIMULATION ARRANGEMENT

(71) Applicant: STIMIT AG, Biel/Bienne (CH)

(72) Inventor: Ronja MÜLLER-BRUHN, Windisch (CH)

(21) Appl. No.: 17/917,410

(22) PCT Filed: Apr. 9, 2021
(86) PCT No.: PCT/EP2021/059260

§ 371 Date: Oct. 6, 2022

(30) Foreign Application Priority Data
Apr. 9, 2020 (CH) 00434/20

(51) Int. Cl. A61B 90/98; A61N 2/00

(52) U.S. Cl. CPC A61B 90/98; A61N 2/006; A61N 2/02

(57) ABSTRACT

A stimulation arrangement for stimulating a target tissue of a patient is disclosed that includes an induction device with an electro-magnetic field generator and a support structure. The electro-magnetic field generator of the induction device includes a coil design configured to generate a spatial electro-magnetic field having a targeted shape. The coil design of the electro-magnetic field generator of the induction device is coupled to the support structure of the induction device. The support structure of the induction device is configured to be positioned at the patient such that the coil design of the electro-magnetic field generator of the induction device is arranged to stimulate the target tissue of the patient by generating the spatial electro-magnetic field. The induction device includes a unique identifier member for distinctly identifying the individual induction device.

### TECHNICAL FIELD

The present invention relates to a stimulation arrangement according to the preamble of independent claim 1 and more particularly to a method of operating such a stimulation arrangement.

Such stimulation arrangement comprising an induction device with an electro-magnetic field generator and a support structure, wherein the electro-magnetic field generator of the induction device comprises a coil design configured to generate a spatial electro-magnetic field having a targeted shape, wherein the coil design of the electro-magnetic field generator of the induction device is coupled to the support structure of the induction device, and wherein the support structure of the induction device is configured to be positioned at the patient such that the coil design of the electro-magnetic field generator of the induction device is arranged to stimulate the target tissue of the patient by generating the spatial electro-magnetic field can be used for stimulating a target tissue such as a Phrenic nerve of a patient.

### BACKGROUND ART

In medical treatments, it is known that for many indications it can be beneficial to activate a target tissue of a patient. For example, in critical care units of hospitals it may be desired to activate the diaphragm of ventilated patients in order to prevent drawbacks of disuse of the diaphragm. It was shown that disuse atrophy of diaphragm muscle fibers occurs already in the first 18-69 hours of mechanical ventilation, and the muscle fiber cross-sections decreased by more than 50% in this time. Thus, it is aimed to activate the diaphragm repeatedly while the patient is given artificially or mechanical respiration such that the functioning of the diaphragm can be upheld, or to activate the diaphragm at least during the weaning period to support effective restoration of independent respiratory function.

For achieving such activation of a target tissue in a patient’s body, it is known that the tissue can be directly stimulated or indirectly activated via stimulation of specific parts of the neural system. For example, the target tissue being a muscular tissue can be activated by providing electric pulses directly to the tissue or to nerves associated to the tissue. More specifically, it is known that the diaphragm can be activated by stimulating the Phrenic nerve, e.g., at the neck of the patient.

In this context, WO 2019/154837 A1 describes an electro-magnetic induction device equipped with an electro-magnetic field generator having a coil design configured to generate a spatial electro-magnetic field with a targeted shape. The device further has a mounting arrangement holding the coil design of the electro-magnetic field generator at the human or animal body and a sensor member configured to detect an activation of the target tissue. It further has an electro-magnetic field adjustment mechanism configured to automatically adjust the position of the electro-magnetic field generated by the coil design, and a calibration unit in communication with the sensor member and with the electro-magnetic field adjustment mechanism. By means of the electro-magnetic field adjustment mechanism and the calibration unit, the device can automatically configure itself to the specific situation given at the patient.

However, even though such known electro-magnetic induction devices allow for an efficient automatic configuration and operation they still have certain drawbacks, particularly, when being used repeatedly. For example, it is difficult to track and/or control the device used in a comparably long term or intermitted application. More specifically, in a care giving institution it can be difficult to know which patient has received which therapy by which device. Also, it can be cumbersome to track the lifetime or durability of the device.

Therefore, there is a need for a stimulation arrangement or process allowing a more efficient control and tracking of applications and therapies provided by operating the stimulation arrangement.

### DISCLOSURE OF THE INVENTION

According to the invention this need is settled by a stimulation arrangement as it is defined by the features of independent claim 1, and by a method of operating such a stimulation arrangement. Preferred embodiments are subject of the dependent claims.

In particular, the invention deals with a stimulation arrangement for stimulating a target tissue of a patient. The stimulation arrangement comprises an induction device with an electro-magnetic field generator and a support structure. The electro-magnetic field generator of the induction device comprises a coil design configured to generate a spatial electro-magnetic field having a targeted shape. The coil design of the electro-magnetic field generator of the induction device is coupled or mounted to the support structure of the induction device. The support structure of the induction device is configured to be positioned at the patient such that the coil design of the electro-magnetic field generator of the induction device is arranged to stimulate the target tissue of the patient by generating the spatial electro-magnetic field. The induction device comprises a unique identifier member configured to distinctly identify the individual induction device.

The patient can be a human or animal being to be treated by electro-magnetic stimulation. Such stimulation can be a beneficial element in a therapeutic treatment of the patient. For example, stimulating a Phrenic nerve for activating the diaphragm can be desired to induce breathing or to assist mechanical ventilation.

The term “target tissue” as used herein can refer to any type of human or animal tissue, including but not limited to skin or muscle tissue. It can more specifically relate to muscle tissue such as a diaphragm or midriff, or tissue of the neural system such as a Phrenic nerve. Thereby, in many applications it is advantageous to repeatedly stimulate the neural system or the nerves and particularly the Phrenic nerve. Such stimulation of the neural system can indirectly activate other tissue such as muscle tissue like a diaphragm or a portion thereof.

The coil design of the electro-magnetic field generator can be or comprise at least two coil units. Each coil unit advantageously has at least one cone shaped or otherwise curved or bulged coil, or at least one small coil, i.e. a coil sufficiently small to generate a sharp electro-magnetic field such as a coil having a diameter of 3 cm or less.

The targeted shape of the electro-magnetic field generated by the coil design can comprise a peak. The electro-magnetic field generator can also be referred to as electro-magnetic field creator. The targeted shape of the electro-magnetic field can be achieved by the electro-magnetic field being a locally constrained, targeted electric field, e.g., having a peak. It can be adapted to be active in a target area being a nerve or tissue area that shall be stimulated by the electromagnetic-field (e.g. the phrenic nerve that shall be stimulated), which can be for example achieved by the peak in the electro-magnetic field (focality area). The targeted shape can generally be any shape of the electro-magnetic field or the time-dependent electric field component that allows to stimulate one or more target tissues or nerves effectively while minimizing other undesired co-stimulation effects of surrounding, above-lying or close-by tissues or nerves. A peak shape is such example, because it maximizes effects in a focality area and minimizes effects outside this area.

The parameters of the voltage or current waveform applied to the coil by a generator affect the temporal characteristics of the electromagnetic field, including pulse shape, amplitude, width, polarity, and repetition frequency; duration of and interval between bursts or trains of pulses; total number of pulses; and interval between stimulation sessions and total number of sessions have, amongst others, an influence on the field strength and determine if and with which intensity or “dose” a target area or target tissue can be activated.

The electro-magnetic field can be generated by the electro-magnetic field generator in single pulses or as a train. Thereby, single pulses relate to the generation of the electro-magnetic field over a comparably short time and with a comparably long interruption between two subsequent pulses. Typically, single pulses are provided at frequencies lower than 10 Hz such as, e.g. at 5 Hz or below, or single pulses are initiated by the user or practitioner. The single pulses can have a temporal width of about 10 to 300 µs. Such pulses can activate nerves and muscles and are identifiable by the patient or by a sensor. In particular, such single pulses may cause a single convulsion of a muscle.

In contrast thereto, when being generated in a train, the electro-magnetic field is either continuously generated or in sequences of pulses comparably quickly following each other. Such pulses can be provided in a frequency range of in between about 15 Hz and about 30 Hz. In particular, a train may achieve to activate a nerve or muscle such that a tetanic contraction or activation is induced. Advantageously, the train is provided by increasing the intensity (field strength) and/or frequency until a target intensity and frequency is achieved (ramp protocol). Like this, sudden convulsion or discomfort can be decreased. All of these parameters are summarized under the term “temporal characteristics” or “temporal parameters” of the electro-magnetic field. These temporal parameters can be adjusted manually via an input interface or be controlled automatically by an adjustment mechanism or control unit.

The temporal characteristics and spatial distribution of the electro-magnetic field can be tuned in such a way that the desired activation (activation feedback) of the target tissue is achieved. Thereby, the activation feedback (signal) refers to a signal that indicates appropriate characteristics of target tissue activation, e.g. a signal that reaches or exceeds a target value (threshold), a signal that exhibits a certain curve pattern or shape, a signal that fulfils a certain algorithm known to represent appropriate target tissue activation in the desired strength, or any combination thereof. The activation feedback (signal) may comprise a feedback in particular about a desired muscle activation strength that shall be reached before the adjustment mechanism stops variation. The appropriate activation feedback signal characteristics can for example be defined by a user via an input interface or be detected by algorithms.

The term “individual induction device” as used in connection with the invention relates to a specific single or unique induction device. In particular, whereas the induction device may be manufactured in batches of plural structurally identical devices, each single one of the induction devices is individual in this sense. Thus, plural individual induction devices can have the same structure but still each of the is individual.

The term “distinctly identify” as used herein relates to recognizing the individual induction device. In particular, such identification allows for recognizing one specific induction device distinct from other possibly structurally identical induction devices.

The support structure can be a mounting arrangement which may be embodied to hold the coil design of the induction device in a specific target position at the human or animal body. In particular, such target position may be a position in which the target tissue such as a targeted portion of the neural system can be reached by the electro-magnetic field generated by the coil design.

The term “holding at” as used in connection with the mounting arrangement can relate to the coil design being in contact with the body or in close distance to it. The position and orientation of the coil design can thereby be predefined or distinct to be appropriate for stimulating the target tissue.

By having the induction device equipped with the unique identifier member, the stimulation arrangement according to the invention allows for an efficient control and tracking of applications and therapies such as the particular beneficial applications and therapies described in more detail below.

Preferably, the stimulation arrangement comprises a digital data storage. Thereby, the digital data storage can be any device, structure or element suitable for storing information in the form of digital data. The digital data can be in any suitable format. The digital data storage can be a single part or a multi part construction. It can be embodied in one component of the simulation arrangement or distributed among plural components. It can be or comprise a read only memory, a read and change memory, or any other structure suitable to store digital data.

By having the digital data storage, the stimulation arrangement can provide information or digital data for evaluation or further processing. More specifically, the digital data storage allows for efficiently making information available which can be used for further processing.

Thereby, the stimulation arrangement preferably is configured to store use data representing a use of the stimulation arrangement in the digital data storage. The use data can be a parameter of the extent of using the stimulation arrangement. A use of the arrangement can be a single or a combination of applications of the stimulation arrangement. For example, the use data can be a counter of the number of stimulations applied by the stimulation arrangement. Storing the use data can be performed by a control unit as described below or by the unique identifier member itself. For example, the control unit can be embodied to retrieve the identification information of the specific stimulation arrangement.

By storing the use data, information about the use of the stimulation arrangement can be made available for evaluation or further processing. For example, by storing the use data, it can be derived if the stimulation arrangement still is in an appropriate condition for further uses. Or, it allows for charging services performed by the stimulation arrangement on the basis of the extent of its use. Such extent may involve the number of applications, a type of application or a combination thereof.

The stimulation arrangement preferably is configured to store life time data representing an operating life time of the induction device in the digital data storage. The term “life time data” in this context relates to data representing any information appropriate to identify the life time of the stimulation arrangement. For example, it may be or comprise a date when the stimulation arrangement was manufactured and/or a date when the stimulation arrangement was first used. By storing the life time data and, thus, making the life time data available it can be considered if the stimulation arrangement still is in shape for further use. For example, as a medical device the stimulation arrangement may be authorized for a certain life time which can be efficiently verified by means of the life time data.

Preferably, the digital data storage comprises an identifier part included in the unique identifier member. Thereby, the identifier part of the digital data storage of the unique identifier member preferably stores unique identifying data representing the individual induction device. The unique identifying data can be a string or a code or the like. Such identifier part, particularly storing the unique identifying data, allows for efficiently and safely identifying the individual induction device. Additionally, the identifier part can store the use data and/or the life time data mentioned above.

The unique identifier member preferably has an electrically erasable programmable read-only memory (EEPROM) comprising the identifier part of the digital data storage. By being equipped with the EEPROM, the unique identifier member of the stimulation arrangement can efficiently and safely be embodied. Particularly, such EEPROM allows for being efficiently being read for verification and tracking purposes.

Preferably, the unique identifier member is included in the coil design of the electro-magnetic field generator. Such arrangement allows for efficiently and safely embodying the unique identifier member.

Preferably, the stimulation arrangement comprises a control unit configured to control operation of the induction device. The control unit can be or comprise a suitable computing device integrated into any of the other components of the stimulation arrangement or being embodied as separate unit. It can also be a distributed unit having components integrated in other components and/or a specific component.

The control unit can be embodied by a digital data processing device or computer. The term “computer” in this connection can relate to any suitable computing device such as laptop computer, a desktop computer, a server computer, a tablet, a smartphone. The term covers single devices as well as combined devices. A computer can, for example, be a distributed system, such as a cloud solution, performing different tasks at different locations. A computer typically involves a processor or central processing unit (CPU), a permanent data storage having a recording media such as a hard disk, a flash memory or the like, a random access memory (RAM), a read only memory (ROM), a communication adapter such as an universal serial bus (USB) adapter, a local area network (LAN) adapter, a wireless LAN (WLAN) adapter, a Bluetooth adapter or the like, and a user interface such as a keyboard, a mouse, a touch screen, a screen, a microphone, a speaker or the like. Computers can be embodied with a broad variety of components as the components listed here.

The control unit allows for efficiently evaluating information about the stimulation arrangement. Like this, operation of the induction device or support structure can be tracked, monitored and/or controlled.

The control unit can be embodied to be coupled to various individual induction devices. Like this, the control unit can be used for controlling plural applications or therapies in parallel. The unique identifier member allows the control unit to identify which one of the individual induction devices is coupled at a time. Like this, it can efficiently control, track or evaluate the use or application of induction devices.

Thereby, the digital data storage preferably comprises a control part included in the control unit. Such control part of the digital data storage allows for efficiently storing data relating to the control and/or use of the individual induction device. When being embodied to be coupled to plural individual induction devices, the control part of the digital data storage can store data for each one of the individual induction devices which may be related by means of information obtained from the unique identifier member. Thus, the control unit preferably is configured to obtain an identification information from the unique identifier member. For example, the control part can store the use data and/or the life time data mentioned above.

Preferably, the stimulation arrangement and, more particularly, its control unit is configured to assign an identification information of the unique identifier member to an individual therapy. Parameters of the therapy can be stored in the control part of the digital data storage. By such configuration, the individual therapy can efficiently be applied when the unique identifier is detected.

The control unit preferably is configured to operate the induction device in accordance with the individual therapy assigned to the identification information. Like this, the individual induction device can be related to one specific therapy.

Preferably, the stimulation arrangement is configured to assign an identification information of the unique identifier member to an individual patient. Like this, the induction device can be related to on specific patient. Thereby, the control unit is configured to operate the induction device in accordance with the individual patient assigned to the identification information. Like this, the induction device can be individualized in terms of a specific patient and a specific therapy.

The stimulation arrangement can be configured to store use data representing a use of the stimulation arrangement in the digital data storage and/or life time data representing an operating life time of the induction device in the digital data storage. The digital data storage can be in the control unit.

Preferably, the support structure of the induction device is configured to be individually adapted to the patient such that the coil design is arranged to stimulate the individual target tissue of the patient. The term “individual target tissue” in this context can relate to a tissue of the individual patient which undergoes a therapy by stimulation of the target tissue. Like this, the support structure can be individualized to specifically suit to the individual patient. For example, the support structure can be embodied to be arranged around a neck of the individual patient, wherein the induction device is positioned and oriented such that the target tissue, e.g. a Phrenic nerve, is within the electro-magnetic field generated by the coil design when operated. Alternatively, the support structure can be positioned at three spots of contact on or at the body of the patient, wherein the three spots can be located relative to each other such that the support structure fits to the individual patient. Particularly in applications requiring multiple therapeutic sessions, the individually adaptable support structure can be beneficial.

In one preferred embodiment, the stimulation arrangement is configured to store support structure data representing an individual adaptation of the support structure of the induction device of the individual patient in the digital data storage. Like this, information about the correct adaptation of the support structure can be provided. For example, this allows to verify if the support structure is correctly adapted for the individual patient.

Thereby, the stimulation arrangement preferably is configured to evaluate the support structure data to identify the individual patient and to automatically adapt the support structure of the induction device in accordance the evaluated support structure data of the identified individual patient.

In another preferred embodiment, the support structure of the induction device has a blocking configuration to block the support structure when being individually adapted. Like this, the individually adaptable support structure can be blocked once individually adapted. Thus, it can efficiently be re-arranged at the patient.

Preferably, the identification information is the unique identifying data stored in the identifier part of the digital data storage.

Preferably, the control unit has a first coupling element and the induction device has a second coupling element corresponding to the first coupling element, wherein the stimulation arrangement is configured to automatically transfer the identification information when the first coupling element of the control unit and the second element of the induction device are connecting. The transfer can be control unit induced or induction device induced. Such first and second coupling elements allow for automatically identifying the individual induction device and to adapt a configuration or parameters of application as soon as the induction device is connected to the control unit. The coupling elements can be embodied as plug and socket, or the like.

Another aspect of the invention relates to a method of operating a stimulation arrangement as described above. The method comprises the steps of: evaluating a unique identifier member of an induction device of the stimulation arrangement; positioning a support structure of the stimulation arrangement at the patient such that a coil design of an electro-magnetic field generator of the induction device of the stimulation arrangement is arranged to stimulate the target tissue by the coil design generating a spatial electro-magnetic field; and operating the induction device of the stimulation arrangement such that the target tissue is stimulated. By this method the effects and benefits described above in connection with the stimulation arrangement according to the invention and its preferred embodiments can efficiently be achieved.

Preferably, evaluating the unique identifier member comprises individually adapting the support structure in accordance with the unique identifier member. Such individual adaptation allows for automatically or verified manual adaptation of the support structure to suit to the situation given at the individual patient associated to the individual induction device.

Preferably, evaluating the unique identifier member comprises charging a use of the stimulation arrangement. For example, the patient or practitioner can be billed in accordance with a number and/or duration of the use of the stimulation arrangement.

Preferably, evaluating the unique identifier member comprises detecting a specific therapy associated to the individual patient and operating the induction is performed in accordance with the detected specific therapy. The specific therapy can be defined by a frequency and/or intensity of the electro-magnetic field generated by the induction device, a duration of generation of the electro-magnetic field, or similar parameters.

### BRIEF DESCRIPTION OF THE DRAWINGS

The stimulation arrangement according to the invention and the method of operating the stimulation arrangement are described in more detail herein below by way of exemplary embodiments and with reference to the attached drawings, in which:

FIG. 1 shows a schematic view of a first embodiment of a stimulation arrangement according to the invention having a first embodiment of an induction device;

FIG. 2 shows a schematic view of a control unit of the stimulation arrangement of FIG. 1;

FIG. 3 shows a schematic view of the induction device of the stimulation arrangement of FIG. 1;

FIG. 4 shows a schematic view of a second embodiment of an induction device of a stimulation arrangement according to the invention; and

FIG. 5 shows a schematic view of a second embodiment of an induction device of a stimulation arrangement according to the invention.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element’s or feature’s relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

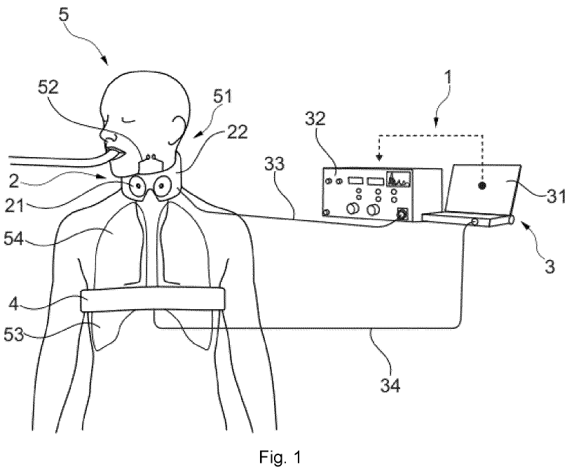

FIG. 1 show an embodiment of a stimulation arrangement 1 according to the invention applying an embodiment of a method of operating the stimulation arrangement 1. The stimulation arrangement 1 is designed for stimulating Phrenic nerves 52 as target tissue of a patient 5 to activate a diaphragm 53 of the patient 5.

The stimulation arrangement 1 comprises an induction device 2, a control unit 3 and a sensor belt 4 as sensor member. The induction device 2 has an electro-magnetic field generator with two conical coil units 21 as coil design and a support structure 22. The control unit 3 comprises a workstation 31 in from of a laptop computer and a stimulator 32. The workstation 31 is connected or coupled to the sensor belt 4 via a sensor cable 34 such that signals sensed by the sensor belt 4 can be transferred to the workstation 31. The stimulator 32 is connected or coupled to the induction device 2 via device cable 33 such that the stimulator 32 can control operation of the induction device 2. The workstation 31 and the stimulator 32 are wirelessly interconnected to transfer data signals and instructions.

The support structure 22 is designed in the shape of a ruff which is arranged around a neck 51 of the patient 5. The coil units 21 have conical coils configured to generate a spatial electro-magnetic field having a targeted shape. They further are coupled or mounted to the support structure 22 such that each of them is located near to one of the two Phrenic nerves 52 of the patient 5. More specifically, the coil units 21 are positioned and oriented at the patient 5 such that each one of the Phrenic nerves 52 is within the electro-magnetic field generated by one of the coil units 21 when operated. Thus, when operating the induction device 2 by supplying electric energy to the coil units 21 the Phrenic nerves 52 are stimulated which, in turn, activates the diaphragm 53 of the patient 5. By activating and de-activating the diaphragm 53 air is provided into and out of a lung 54 of the patient 5.

The workstation 31 has a data storage, in which operation parameters of specific therapies of specific induction devices 2 are stored. In particular, sets of parameters such as electric energy intensities, temporal widths and the like are stored which together define a protocol of the respective therapy. The protocol can provide sequences of electro-magnetic pulses or trains shaped and structured in accordance with the intended therapy.

By receiving the data signals provided by the sensor belt 4, the workstation 31 evaluates the activation of the diaphragm 53 and, if required controls the stimulator 32 to adjust operation of the induction device 2. Like this, a feedback loop is established which allows for accurately controlling the stimulation arrangement 1 for efficiently activating the diaphragm 53 and inducing breathing or assisting ventilation.

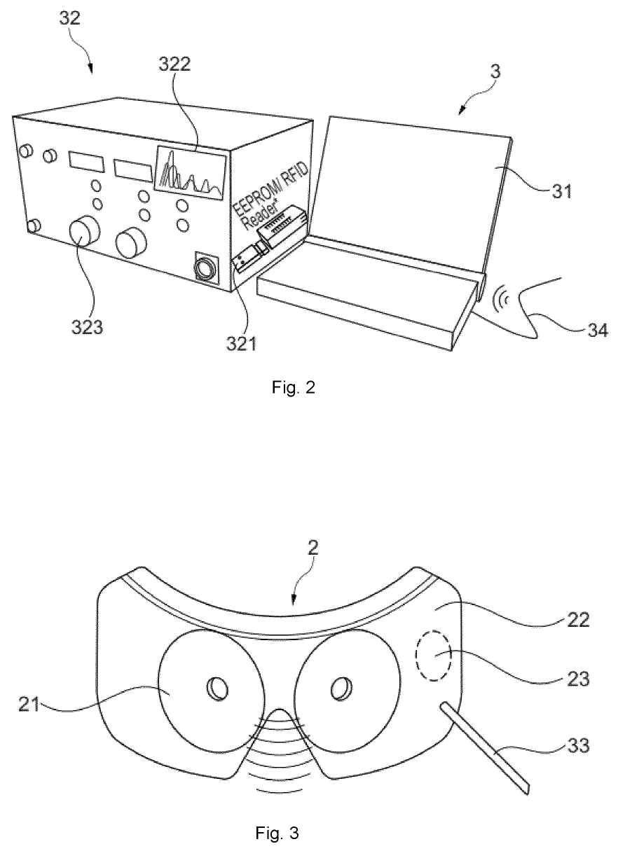

In FIG. 2 the control unit 3 is shown in more detail. The stimulator 32 is provided with a display 322 showing operation of the induction device 2 and a set of control buttons 323. It further is equipped with an EEPROM reader 321 as a portion of a first coupling element.

FIG. 3 shows the induction device 2 in more detail. The induction device comprises an electrically erasable programmable read-only memory 23 (EEPROM) as unique identifier member and as a portion of a second coupling element. The EEPROM 23 together with the data storage of the workstation 31 and a data storage of the stimulator 32 establish a digital data storage of the stimulation arrangement 1. More specifically, the EEPROM 23 is an identifier part of the digital data storage of the stimulation arrangement 1, and the data storage of the workstation 31 together with the data storage of the stimulator 32 are a control part of the digital data storage of the stimulation arrangement 1.

In the EEPROM 23 unique identifying data is stored. The unique identifying data represents identification information of the individual induction device 2 to which the EEPROM 23 is mounted. When the first coupling element of the control unit 3 and the second coupling element of the induction device 2 are connected, the unique identifying data is automatically transferred from the EEPROM 23 to the control unit 3. More specifically, the EEPROM reader 321 of the stimulator 32 reads the unique identifying data in the EEPROM 23 of the induction device 2 as soon as the first and second coupling elements are engaged. The unique identifying data is then also transferred to the workstation 31.

In the workstation 31 the unique identifier data is further processed. Thereby, the workstation 31 creates or updates use data representing the use of the stimulation arrangement 1 in its data storage. In particular, a number of operations of the induction device 2 is stored in the data storage of the workstation 31 as portion of the use data. The use data is involved in charging a practitioner of the stimulation arrangement 1. More specifically, the stimulation arrangement 1 is rented by the practitioner and he is billed on the basis of the number and type of uses applying the stimulation arrangement 1.

Further, based on the unique identifier data the workstation 31 creates or updates life time data representing an operating life time of the induction device 2 in its data storage. Thereby, the workstation 31 controls the life time of the induction device 2 and prevents use of it after a certain life time in order to assure proper operation of the system.

For operating the induction device 2, the workstation 31 adjusts the stimulator 32 in accordance with a specific therapy provided to the patient 5. In turn, as mentioned above, the stimulator 32 operates the induction device 2 such that the coil units 21 generate the electro-magnetic field to coordinately stimulate the two Phrenic nerves 52 of the patient 5 for activating the diaphragm 53 of the patient 5. For example, like this assisting mechanical ventilation to prevent diaphragm disuse atrophy can be achieved.

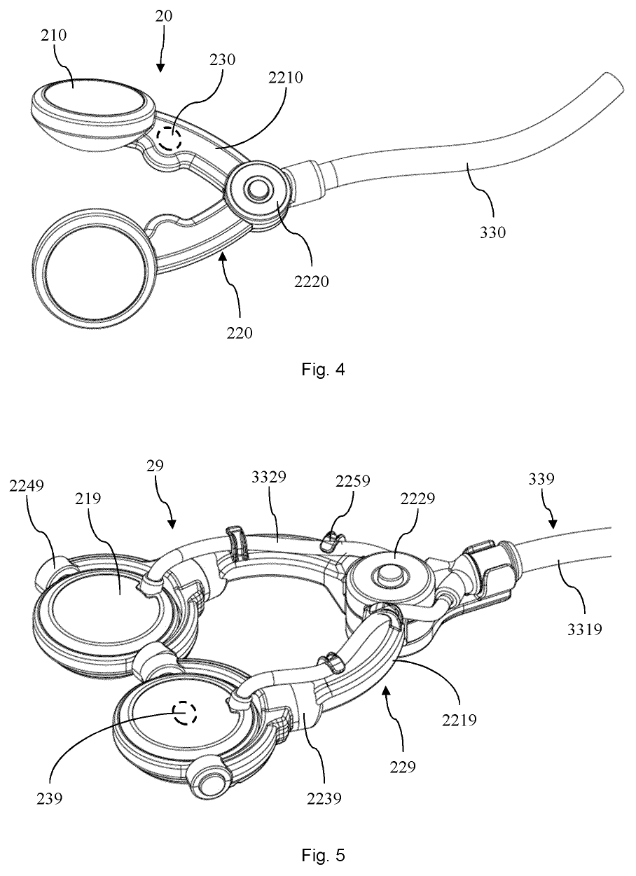

In FIG. 4 a second embodiment of an induction device 20 is shown. The second induction device 20 can be used with or operated by the same or a similar control unit 3 as the first induction device 2 described above. The induction device 20 has two coil units 210 with a rounded forward face configured to be positioned at the patient 5 to stimulate the Phrenic nerves 52. Each of the coil units 210 comprises a housing for accommodating an associated non-flat or conical coil winding and for providing contact surfaces to be positioned at the patient 5. By such housings the coil windings of the coil units 210 can be encased and protected as well as safely positioned and precisely oriented allowing to assure correct positioning and orienting of the coil windings relative to the patient 5 and relative to each other.

The induction device 20 further comprises a support structure 220 coupled to the coil units 210. Advantageously, the coil units 210 can be detachably coupled to the support structure 220 to be replaced, e.g. disposed, in case needed. The support structure 220 is releasably connected to a current supply cable 330 for coordinatedly providing current to the coil units 210 for generating electro-magnetic fields such that the Phrenic nerve 52 of the patient 5 are stimulated and a diaphragm 53 of the patient 5 is homogeneously activated.

The support structure 220 comprises two leg portion 2210 connected by a hinge portion 2220. The leg portion 2210 are realized as flat rod elements having a curved shape. Each one of the coil units 210 is releasebly coupled to one end of one of the leg portions 2210. The hinge portion 2220 is arranged at an opposite end of the leg portions 2210 and flexibly couples the leg portions 2210 at their opposing ends. One of the leg portions 2210 is provided with an EEPROM 230 which stores unique identifier data.

The hinge portion 2220 is configured as a pivot bearing such that the leg portions 2210 can be pivoted around a common articulation axis of the bearing that runs through the centre of the hinge portion 2220. Thus, the leg portions 2210 can be swivelled around the articulation axis in a common two-dimensional plane. The hinge portion 2220 allows for adjusting a distance between the coil units 210 to adapt the position and orientation of thereof according to a desired target configuration of the support structure 20 depending on the situation given at the patient 5.

The curved shape of the leg portions 2210 assures that in any pivoting position a free space is provided between the hinge portion 2220 and the leg portions 2210. The free space provides ample access to a front area of the neck of a patient that is commonly important for treatment of the patient such as a tracheotomy. The leg portions 2210 are curved in one geometrical plane. Once the specific pivoting position is adjusted a button on the hinge portion 2220 is pushed which prevents any movement of leg portions 2210 and the coil units 210 relative to each other. For example, the blocking can be provided by a mechanical mechanism or an electrical arrangement. In the present embodiment, the button blocks movement of the leg portions 2210 by providing a form fit within the hinge portion 2220 in a pushed position. This establishes a blocking configuration which blocks the support structure 20 when being individually adapted. The blocking configuration prevents changes of the configuration of the support structure 220 after being individually adjusted, which guarantees adequate stimulation of the Phrenic nerves 52 by the induction device 20. Thus, the induction device 20 can be removed from the patient 5 and replaced at a later point in time without the need of recalibration, repositioning and/or reorienting of the coil units 210.

In use, the induction device 20 rests on the body surface of the patient 5 only with the three forward surfaces located at the coil units 210 and at the support structure 230. This enables stable and well-defined positioning at the patient independent of an individual surface landscape of the patient’s body while space constraints can be overcome and the respiration promoting apparatus can be carried by the patient conveniently and pain-free. Further, the geometry of the support structure 220 determines a target configuration of the induction device 20 and determines a defined location and orientation of the coil units 210 generating the electro-magnetic fields for activating the diaphragm 53. This results in efficient stimulation of both Phrenic nerves 52 and avoids co-stimulation effects of tissue in the vicinity of the two Phrenic nerves 52.

Since the individual adaptation of the support structure 220 of the induction device is specific for the one single patient 5, the unique identifier data transferred from the induction device 20 to the workstation 31 via the EEPROM reader 321 also identifies the individual patient 5. Like this, the workstation 31 controls the stimulator 32 to operate the induction device 20 in accordance with a protocol stored in the data storage of the workstation 31. In particular, the protocol is associated to the patient 5 and to the therapy required by him. Like this, the control unit 3 can automatically identify the individual patient 5 and provide the appropriate treatment.

FIG. 5 shows a third embodiment of a induction device 29 which can be used with or operated by the same or a similar control unit 3 as the first and second induction devices 2, 20 described above. The third induction device 29 is generally configured analogue to the second induction device 20 but includes additional or alternative features as described in the following.

The two coil units 219 are coupled to the support structure 229 via U-like shaped pivoting couplers 2249. The U-shape is realized by a stem portion 2239 and two symmetrically curved branch portions of the pivoting couplers 2249. Each one of the stem portions 2239 of the pivoting couplers 2249 is mounted to an end of one of the leg portions 2219. One of the coil units 219 is provided with an EEPROM 239 in which unique identifier data is stored.

A main portion 3319 of a current supply cable 339 is releasably held in one of plural clip mountings 2259 arranged at an extension plate extending from the hinge portion 2229. The supply cable 339 has two sub portions 3329, which run along the support structure 229 and provide current pulses or trains to the coil units 219. The sub portions 3329 are detachably fixed to the leg portions 2219 of the support structure 229 by respective clip mountings 2259. The sub portions 3329 are connected to the coil units 219 by common connectors. The complete supply cable 339 can be detached from the support structure 229 and from the coil units 219. Thus, it can be used with a different support structure 229 and different coil units 219 and the induction device 29 can easily be modified for different use situations. For example, the support structure 229 can be embodied as disposable associated to a specific patient 5 and to be replaced after treatment of the specific patient 5.

Each of the pivoting couplers 2249 is pivotably mounted to one of the leg portions 2219 via its stem portion 2239. Depending on an adjustment angle of the leg portions 2219 relative to each other at the hinge portion 2229 the coil units 219 can also be adjusted. Thereby, the coil units 219 are also pivotably mounted between the branch portions such that they can be pivoted relative to the respective pivoting coupler.

This description and the accompanying drawings that illustrate aspects and embodiments of the present invention should not be taken as limiting-the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below.

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1-23. (canceled)

24. A stimulation arrangement for stimulating a target tissue of a patient, the stimulation arrangement comprising:
an induction device with an electro-magnetic field generator and a support structure, wherein
the electro-magnetic field generator of the induction device comprises a coil design configured to generate a spatial electro-magnetic field having a targeted shape,
the coil design of the electro-magnetic field generator of the induction device is coupled to the support structure of the induction device,
the support structure of the induction device is configured to be positioned at the patient such that the coil design of the electro-magnetic field generator of the induction device is arranged to stimulate the target tissue of the patient by generating the spatial electro-magnetic field, and
the induction device comprises a unique identifier member distinctly identifying the individual induction device.

25. The stimulation arrangement of claim 24, comprising a digital data storage, preferably configured to store use data representing a use of the stimulation arrangement in the digital data storage.

26. The stimulation arrangement of claim 25, configured to store lifetime data representing an operating lifetime of the induction device in the digital data storage.

27. The stimulation arrangement of claim 25, wherein the digital data storage comprises an identifier part included in the unique identifier member.

28. The stimulation arrangement of claim 27, wherein the identifier part of the digital data storage of the unique identifier member stores unique identifying data representing the individual induction device.

29. The stimulation arrangement of claim 27, wherein the unique identifier member has an electrically erasable programmable read-only memory comprising the identifier part of the digital data storage.

30. The stimulation arrangement of claim 24, wherein the unique identifier member is included in the coil design of the electro-magnetic field generator.

31. The stimulation arrangement of claim 24, comprising a control unit configured to control operation of the induction device.

32. The stimulation arrangement of claim 31, wherein the digital data storage comprises a control part included in the control unit.

33. The stimulation arrangement of claim 31, wherein the control unit is configured to obtain an identification information from the unique identifier member.

34. The stimulation arrangement of claim 24, configured to assign an identification information of the unique identifier member to an individual therapy.

35. The stimulation arrangement of 34, comprising a control unit configured to control operation of the induction device, wherein the control unit is configured to operate the induction device in accordance with the individual therapy assigned to the identification information.

36. The stimulation arrangement of claim 24, configured to assign an identification information of the unique identifier member to an individual patient.

37. The stimulation arrangement of claim 36, comprising a control unit configured to control operation of the induction device, wherein the control unit is configured to operate the induction device in accordance with the individual patient assigned to the identification information.

38. The stimulation arrangement of claim 25, configured to store use data representing a use of the stimulation arrangement in the digital data storage.

39. The stimulation arrangement of claim 25, configured to store lifetime data representing an operating lifetime of the induction device in the digital data storage.

40. The stimulation arrangement of claim 24, wherein the support structure of the induction device is configured to be individually adapted to the patient such that the coil design is arranged to stimulate the individual target tissue of the patient.

41. The stimulation arrangement of claim 40, configured to store life time data representing an operating life time of the induction device in the digital data storage and configured to store support structure data representing an individual adaptation of the support structure of the induction device of the individual patient in the digital data storage, wherein the stimulation arrangement preferably is configured to evaluate the support structure data to identify the individual patient and to automatically adapt the support structure of the induction device in accordance the evaluated support structure data of the identified individual patient.

42. The stimulation arrangement of claim 41, wherein the support structure of the induction device has a blocking configuration to block the support structure when being individually adapted.

43. The stimulation arrangement of claim 33,
wherein the unique identifier member has an electrically erasable programmable read-only memory comprising the identifier part of the digital data storage and wherein the identification information is the unique identifying data stored in the identifier part of the digital data storage, and/or
wherein the control unit has a first coupling element and the induction device has a second coupling element corresponding to the first coupling element, wherein the stimulation arrangement is configured to automatically transfer the identification information when the first coupling element of the control unit and the second element of the induction device are connecting.
